(19) United States
(12) Patent Application Publication
(10) Pub. No.: US 2004/0213802 A1
(43) Pub. Date: Oct. 28, 2004

(54) Gene of novel corynebacterium diphtheriae antigen

(76) Inventor: Masaaki Iwaki, Tokyo (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 10/487,550

(22) PCT Filed: Feb. 27, 2002
(86) PCT No.: PCT/JP02/01776

(30) Foreign Application Priority Data
Aug. 24, 2001 (JP) 2001-253931

(51) Int. Cl. A61K 39/02; C07H 21/04; C12N 1/21; C07K 14/34

(52) U.S. Cl. 424/190.1; 536/23.7; 435/69.3; 435/252.3; 435/320.1; 530/350

(57) ABSTRACT

It is intended to provide an antigen specific to Corynebacterium diphtheriae which is usable in developing vaccines for protecting/preventing infection with diphtheria. A gene encoding the following protein (a) or (b): (a) a protein comprising the amino acid sequence represented by SEQ ID NO:1 as described below:

(SEQ ID NO:1)

| | |
|---|---|
| M F R R S L A S L A | A V A V I S G V V V |
| A P A N A V T V T V | N N M T C T I K L T |
| P E E T N F I P L S | S P V M L T K E K A |
| A F L K N Q Y G E T | Q L S A L D A E I K |
| K K E K E L T E L E | P N A A G R E M L T |
| S E L K E K K K R F | T A N K K F S D A L |
| D A C I A G E N Y D | S S K P N G P K D P |
| N G P K K P G D S D | Q S G G S E K P N G |
| P K D P G G S E K P | N D P K H P E V E R |
| A L P S T N G A V I | G A I V A V L G I L |
| A A A L P V I K S I | L R A L L P; |

and (b) a protein comprising an amino acid sequence derived from the amino acid (a) by deletion, substitution or addition of one to several amino acids and having a C. diphtheriae antigenic activity.

### TECHNICAL FIELD

The present invention relates to Corynebacterium diphtheriae antigen gene and to protein encoded thereby.

### BACKGROUND OF THE INVENTION

Diphtheria is a serious infectious disease, belonging to group 2 of the Infectious Diseases Prevention Law, caused by Corynebacterium diphtheriae. Toxoid vaccine where diphtheria toxin which is a main virulence factor is chemically modified is the first one which was developed as vaccine for diphtheria. Since it was very effective for prevention of the onset of the disease, it has been continuously used now showing an excellent result. On the other hand, necessity for the study concerning other virulence factors has become diluted and, as a result, studies in this field have been delayed.

Incidentally, although conventional toxoid vaccine is able to defend the onset of the disease in the person who was infected by diphtheria, it cannot defend or prevent a person from being infected by diphtheria.

Since diphtheria is a serious infectious disease, quick diagnosis at an early stage is demanded; however, as a reliability of a PCR method and a quick diagnostic method have not been established yet, it is essential for diagnosis of diphtheria at present to incubate the microbe separated from the diseased part and that requires for several days. It is important for an early treatment to shorten the above period.

With regard to references for diphtheria infection, “Hiroko Sato and Motohide Takahashi, Vaccine Handbook 11. Diphtheria Toxoid edited by the Gakuyukai of the National Institute of Preventive Medicine, published by Maruzen, page 71, 1994”, and “Mattos-Guaralde, A., et al., Cell Surface Components and Adhesion in Corynebacterium diphtheriae”, Microbes and Infection, (2000) 2, 1507-1512”, etc. may be exemplified.

### DISCLOSURE OF THE INVENTION

In view of the above, if vaccine which defends and prevents the infection of diphtheria is developed, its utility is high. In addition, if antigen which is specific to Corynebacterium diphtheriae is specified, its application as a quick diagnostic agent for diphtheria can be expected. If a novel quick diagnostic method is established, its utility is high. Moreover, antigen which is specific to Corynebacterium diphtheriae is able to be a very effective tool for an epidemiologic search of diphtheria. In fact, there are many people having high antibody titer to diphtheria toxin and, by using a novel antigen which is specific to Corynebacterium diphtheriae, it is able to judge whether that is caused by infection of Corynebacterium diphtheriae or by inoculation of vaccine using diphtheria toxoid.

Antigen and virulence factor for Corynebacterium diphtheriae have been rarely known besides the above-mentioned diphtheria toxoid. If a novel antigen is found, that will result in resolution of the above-mentioned matter.

### BRIEF DESCRIPTION OF THE DRAWINGS

FIG. 1 shows the result of analysis by Western blotting using an anti-PW8 strain polyclonal antibody from a gene library of PW8 strain. In the drawing, the left lane shows the result obtained from total cell lysate of PW8 strain while the right lane shows the result obtained from culture supernatant of cultured PW8 strain.

FIG. 2 shows the result of analysis of a PCR-cloned genetic product (excluding the signal sequence parts) by Western blotting using an anti-PW8 strain polyclonal antibody.

### BEST MODE FOR CARRYING OUT THE INVENTION

As a result of an intensive study, the present inventor has achieved the following invention.

Thus, as the first characteristic feature, the present invention relates to a gene which encodes the protein of the following (a) or (b):

(a) Protein comprising an amino acid sequence represented by the following SEQ ID NO: 1.

| | | | |
|---|---|---|---|
| M F R R S L A S L A | A V A V I S G V V V | A P A N A V T V T V | (SEQ ID NO: 1) |
| N N M T C T I K L T | P E E T N F I P L S | S P V M L T K E K A | |
| A F L K N Q Y G E T | Q L S A L D A E I K | K K E K E L T E L E | |
| P N A A G R E M L T | S E L K E K K K R F | T A N K K F S D A L | |
| D A C I A G E N Y D | S S K P N G P K D P | N G P K K P G D S D | |
| Q S G G S E K P N G | P K D P G G S E K P | N D P K H P E V E R | |
| A L P S T N G A V I | G A I V A V L G I L | A A A L P V I K S I | |
| L R A L L P | | | |

(b) Protein comprising an amino acid sequence derived from the amino acid (a) by deletion, substitution or addition of one to several amino acids and having a Corynebacterium diphtheriae antigenic activity.

The present invention further relates to DNA which encodes a polypeptide of the following (a) or (b):

(a) Polypeptide comprising an amino acid sequence of from the 25th to the 216th or from the 130th to the 190th members from N terminal in an amino acid sequence represented by SEQ ID NO: 1;

(b) Polypeptide comprising an amino acid sequence derived from the polypeptide (a) by deletion, substitution or addition of one to several amino acids and having a Corynebacterium diphtheriae antigen.

Further, as the second characteristic feature, the present invention relates to a gene which comprises a DNA of the following (a) or (b):

(a) DNA comprising a nucleotide sequence (having open reading frame of 651 bp) represented by the following SEQ ID NO: 2 (SEQ ID NO: 2)

| | | | |
|---|---|---|---|
| A T G T T C A G G C | G T T C A C T C G C | G T C C C T C G C T | (SEQ ID NO: 2) |
| G C C G T C G C T G | T C A T C T C T G G | T G T T G T T G T T | |
| G C G C C A G C G A | A T G C C G T G A C | T G T C A C G G T C | |
| A A T A A T A T G A | C C T G C A C C A T | T A A G C T C A C T | |
| C C T G A A G A A A | C T A A T T T T A T | A C C C T T G A G T | |
| T C G C C G G T A A | T G C T C A C C A A | G G A A A A A G C T | |
| G C T T T T C T C A | A A A A T C A A T A | C G G A G A A A C A | |
| C A G C T G T C T G | C C C T T G A C G C | G G A A A T A A A G | |
| A A G A A A G A A A | A G G A A C T G A C | T G A G C T C G A A | |
| C C T A A C G C G G | C G G G G A G G G A | G A T G T T G A C G | |
| A G C G A G C T G A | A A G A G A A A A A | G A A G A G G T T C | |
| A C C G C C A A C A | A A A A A T T C A G | T G A T G C G C T G | |
| G A T G C G T G C A | T C G C A G G T G A | G A A C T A C G A C | |
| T C G A G T A A G C | C C A A T G G C C C | A A A G G A T C C C | |
| A A T G G C C C G A | A G A A G C C C G G | T G A C T C G G A T | |
| C A G T C C G G T G | G C T C G G A G A A | G C C C A A T G G C | |
| C C A A A G G A T C | C C G G T G G C T C | G G A G A A G C C C | |
| A A T G A C C C G A | A G C A T C C C G A | A G T C G A A C G C | |
| G C T C T G C C T T | C G A C C A A C G G | A G C A G T A A T C | |
| G G C G C C A T C G | T C G C G G T T C T | C G G C A T C T T G | |
| G C C G C C G C T C | T G C C T G T C A T | T A A G T C G A T A | |
| C T G C G G G C T C | T C C T G C C G T A | A | |

(b) DNA which hybridizes to the DNA comprising the nucleotide sequence (a) under a stringent condition and encodes a protein having a Corynebacterium diphtheriae antigenic activity.

Furthermore, the present invention relates to DNA of the following (a) or (b):

(a) In the DNA comprising the nucleotide sequence represented by SEQ ID NO: 2, a DNA which encodes a polypeptide comprising an amino acid sequence of from the 25th to the 216th or from the 130th to the 190th members from N terminal in the amino acid sequence represented by SEQ ID NO: 1;

(b) DNA which hybridizes to the DNA comprising the nucleotide sequence (a) under a stringent condition and encodes a polypeptide having a Corynebacterium diphtheriae antigen activity.

The present invention still further relates to protein or polypeptide encoded by such a gene or DNA.

A product which is presumed from the gene of the present invention is a protein (presumed molecular weight: 22,797) comprising 216 amino acids represented by SEQ ID NO: 1. It is presumed that from N terminal to the 24th alanine is a signal peptide. After cleavage of the signal peptide, an N terminal amino acid is valine and a presumed molecular weight is 20,345.

In addition, as a result of analysis using a function of “Peptide Structure” of gene analysis software “GCG”, it was presumed that a polypeptide comprising from 130th to 190th members from N terminal of the above-mentioned amino acid sequence was the part which has the strongest antigenicity. With regard to the algorithm of the GCG, refer to Jameson and Wolf, (1988), CABIOS, 4(1); 181-186.

Accordingly, in view of giving a Corynebacterium diphtheriae antigen activity, it is preferred that deletion or substitution of one to several amino acid(s) in the gene encoding a protein comprising the amino acid sequence represented by SEQ ID NO: 1 of the present invention takes place at the part of other than from the 130th to the 190th members from N terminal of the said amino acid sequence. Incidentally, “Corynebacterium diphtheriae antigenic activity” in the present specification means an activity which is able to significantly conduct a specific bond to antigen of Corynebacterium diphtheriae serum etc.

As will be shown in the following Example, the gene or the DNA of the present invention is able to be prepared by cloning from a gene library derived from Corynebacterium diphtheriae by means of Western blotting, PCR cloning, etc. using a polyclonal antibody to the said bacillus whereby the nucleotide sequence is determined. Although there is no particular limitation for the Corynebacterium diphtheriae strain, a PW8 strain may be exemplified.

It is also possible to prepare by chemical synthesis, etc. using a known method in the technical field on the basis of sequence information disclosed in the present specification. Persons skilled in the art can be easily conduct deletion, substitution or addition of one to several amino acid(s) in a specific amino acid sequence using known method in the technical field.

In the present specification, a stringent condition for hybridization to specific gene or DNA may be appropriately established by persons skilled in the art.

An example of DNA which hybridizes to the gene or the DNA of the present invention under a stringent condition and also encodes the protein which is a Corynebacterium diphtheriae antigen is a DNA where homology to such a gene or DNA is not less than 80%, preferably not less than 90% and, more preferably, not less than 95%.

DNA molecules in which various sequences which have been known among persons skilled in the art in the gene recombination operations, for example, regulatory factor such as promoter and enhancer; restriction enzyme site; and selective marker gene are bonded to the gene or the DNA of the invention are also within a scope of the present invention. Further, expression vehicle such as a recombinant vector containing the gene or the DNA of the present invention or the above-mentioned DNA molecule, and transformant such as cell and eukaryote containing the expression vehicle and being transformed thereby are also covered by the present invention. Such DNA molecule, expression vehicle and transformant can be easily prepared by the known method in the technical field.

The protein or the polypeptide of the present invention can be easily prepared by a known method in the technical field using such as the above-mentioned expression vehicle, transformant. For example, it can be prepared by expressing the gene or the DNA of the present invention by incubation of a transformant followed by recovering it. Accordingly, the present invention relates to a preparation method thereof.

The present invention further relates to a polyclonal antibody or a monoclonal antibody to such a protein or polypeptide. Such an antibody can be produced by the method which has been known among persons skilled in the art.

### EXAMPLES

The present invention will now be illustrated in detail by way of the following Examples, however, those Examples do not limit the technical scope of the present invention by any means.

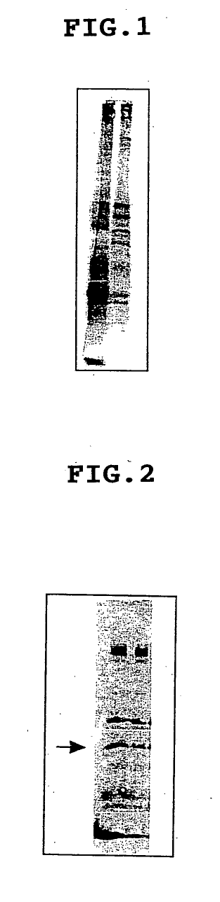

Firstly, it was checked whether Corynebacterium diphtheriae has unknown antigens. When total cells of a Corynebacterium diphtheriae vaccine strain PW8 were lysed in SDS and analyzed by means of an SDS-PAGE and a Western blotting, many bands reacting with anti-PW8 strain polyclonal antibody (rabbit anti-PW8 total cell antiserum) were detected (FIG. 1). From the above, it was clarified that, in Corynebacterium diphtheriae, there were many antigens other than diphtheria toxin. Incidentally, the SDS-PAGE was carried out using 12% polyacrylamide gel and other conditions were principally the same as those according to Laemmli, U. K., (1970), Nature, 227, 680-685.

Next, in order to isolate the gene of those antigens, incubation was carried out in an BHI liquid medium for two days so that total DNA of the PW8 strain was partially digested with a restriction enzyme Sau 3AI whereupon fragments of about 2 to 4 kb were isolated. They were connected to pUC 19 vector having a lac promoter to prepare a gene library comprising about 5,000 clones. After that, 49 clones reacting with an anti-PW8 strain polyclonal antibody were selected from the library using a rabbit anti-PW8 strain total cell anti-serum. When those selected clone products were analyzed by a Western blotting, they showed bands where molecular weights were from 20,000 to 100,000. Nucleotide sequence of the DNA derived from the PW8 strain contained therein was determined and DNA which is one of them represented by SEQ ID NO: 1 was subjected to a PCR cloning together with estimating its ORF whereupon it showed a strong antigenicity (FIG. 2).

The Western blotting was carried out according to the standard method (Harlow, E. and Lane, D., (1988), Antibodies—A Laboratory Manual, Cold Spring Harbor Laboratory Press). Incidentally, an anti-PW8 strain polyclonal antibody in 500-fold was used as a primary antibody while, as a secondary antibody, a goat anti-rabbit IgG labeled with alkaline phosphatase (Tago), etc. were used.

Determination of a nucleotide sequence was also carried out according to the standard method where, using sequencer of Models 373, 377, 310 and 3100 of Applied Biosystems and also using Dye Terminator FS kit and Big Dye Terminator kit, an operation was carried out according to the manuals for those kits and sequencers.

The PCR was also carried out according to the standard method using Thermal Cycler type MP of Takara Shuzo or Model 2400 of Perkin Elmer and using ExTaq polymerase of Takara Shuzo.

The condition for the PCR was 98° C. for 2 minutes and then a cycle of 98° C. for 10 seconds and 68° C. for 1 minute was repeated for 30 cycles followed by cooling to 4° C.

The DNA containing the gene of the present invention (clone 1-1 ORF 2) was deposited in the International Patent Organism Depository, the National Institute of Advanced Industrial Science and Technology, Japan on Aug. 20, 2001 with an accession number of FERM P-18476. That was then transferred to the international deposition on Feb. 12, 2002 with an accession number of FERM BP-7885.

### INDUSTRIAL APPLICABILITY

In accordance with the present invention, amino acid sequence and nucleotide sequence of protein or polypeptide which are antigens specific to Corynebacterium diphtheriae were specified. The protein or polypeptide is useful as an effective ingredient for vaccine for prevention of infection of diphtheria, as a quick diagnostic agent and as a very effective tool for an epidemiological investigation for diphtheria. In addition, an antibody to the said protein or polypeptide is also useful as an effective ingredient for a pharmaceutical composition for prevention of infection of diphtheria.

1. Gene which encodes the protein of the following (a) or (b):
(a) Protein comprising an amino acid sequence represented by the following SEQ ID No: 1.

| | | | |
|---|---|---|---|
| M F R R S L A S L A | A V A V I S G V V V | A P A N A V T V T V | (SEQ ID NO: 1) |
| N N M T C T I K L T | P E E T N F I P L S | S P V M L T K E K A | |
| A F L K N Q Y G E T | Q L S A L D A E I K | K K E K E L T E L E | |
| P N A A G R E M L T | S E L K E K K K R F | T A N K K F S D A L | |
| D A C I A G E N Y D | S S K P N G P K D P | N G P K K P G D S D | |
| Q S G G S E K P N G | P K D P G G S E K P | N D P K H P E V E R | |
| A L P S T N G A V I | G A I V A V L G I L | A A A L P V I K S I | |
| L R A L L P | | | |

(b) Protein comprising an amino acid sequence derived from the amino acid (a) by deletion, substitution or addition of one to several amino acids and having a Corynebacterium diphtheriae antigenic activity.

2. DNA which encodes a polypeptide of the following (a) or (b):
(a) Polypeptide comprising an amino acid sequence of from the 25th to the 216th members from N terminal in an amino acid sequence represented by SEQ ID NO: 1;
(b) Polypeptide comprising an amino acid sequence derived from the polypeptide (a) by deletion, substitution or addition of one to several amino acids and having a Corynebacterium diphtheriae antigenic activity.

3. DNA which encodes a polypeptide of the following (a) or (b):
(a) Polypeptide comprising an amino acid sequence of from the 130th to the 190th members from N terminal in an amino acid sequence represented by SEQ ID NO: 1;
(b) Polypeptide comprising an amino acid sequence derived from the polypeptide (a) by deletion, substitution or addition of one to several amino acids and having a Corynebacterium diphtheriae antigenic activity.

4. Gene which comprises a DNA of the following (a) or (b):
(a) DNA comprising a nucleotide sequence represented by the following SEQ ID NO: 2

| | | | |
|---|---|---|---|
| A T G T T C A G G C | G T T C A C T C G C | G T C C C T C G C T | (SEQ ID NO: 2) |
| G C C G T C G C T G | T C A T C T C T G G | T G T T G T T G T T | |
| G C G C C A G C G A | A T G C C G T G A C | T G T C A C G G T C | |
| A A T A A T A T G A | C C T G C A C C A T | T A A G C T C A C T | |
| C C T G A A G A A A | C T A A T T T T A T | A C C C T T G A G T | |
| T C G C C G G T A A | T G C T C A C C A A | G G A A A A A G C T | |
| G C T T T T C T C A | A A A A T C A A T A | C G G A G A A A C A | |
| C A G C T G T C T G | C C C T T G A C G C | G G A A A T A A A G | |
| A A G A A A G A A A | A G G A A C T G A C | T G A G C T C G A A | |
| C C T A A C G C G G | C G G G G A G G G A | G A T G T T G A C G | |
| A G C G A G C T G A | A A G A G A A A A A | G A A G A G G T T C | |
| A C C G C C A A C A | A A A A A T T C A G | T G A T G C G C T G | |
| G A T G C G T G C A | T C G C A G G T G A | G A A C T A C G A C | |
| T C G A G T A A G C | C C A A T G G C C C | A A A G G A T C C C | |
| A A T G G C C C G A | A G A A G C C C G G | T G A C T C G G A T | |
| C A G T C C G G T G | G C T C G G A G A A | G C C C A A T G G C | |
| C C A A A G G A T C | C C G G T G G C T C | G G A G A A G C C C | |
| A A T G A C C C G A | A G C A T C C C G A | A G T C G A A C G C | |
| G C T C T G C C T T | C G A C C A A C G G | A G C A G T A A T C | |
| G G C G C C A T C G | T C G C G G T T C T | C G G C A T C T T G | |
| G C C G C C G C T C | T G C C T G T C A T | T A A G T C G A T A | |
| C T G C G G G C T C | T C C T G C C G T A | A | |

(b) DNA which hybridizes to the DNA comprising the nucleotide sequence (a) under a stringent condition and encodes a protein having a Corynebacterium diphtheriae antigenic activity.

5. DNA of the following (a) or (b):
(a) In the DNA comprising the nucleotide sequence represented by SEQ ID NO: 2, a DNA which encodes a polypeptide comprising an amino acid sequence of from the 25th to the 216th members from N terminal in the amino acid sequence represented by SEQ ID NO: 1;
(b) DNA which hybridizes to the DNA comprising the nucleotide sequence (a) under a stringent condition and encodes a polypeptide having a Corynebacterium diphtheriae antigenic activity.

6. DNA of the following (a) or (b):
(a) In the DNA comprising the nucleotide sequence represented by SEQ ID NO: 2, a DNA which encodes a polypeptide comprising an amino acid sequence of from the 130th to the 190th members from N terminal in the amino acid sequence represented by SEQ ID NO: 1;
(b) DNA which hybridizes to the DNA comprising the nucleotide sequence (a) under a stringent condition and encodes a polypeptide having a Corynebacterium diphtheriae antigenic activity.

7. Protein or polypeptide which is encoded to the gene or the DNA mentioned in any of claims 1 to 6.

8. An expression vehicle containing the gene or the DNA mentioned in any of claims 1 to 6.

9. A transformant which is transformed by the expression vehicle mentioned in claim 8.

10. A method for the preparation of a protein or a polypeptide, comprising culturing the transformant mentioned in claim 9 so that the gene or the DNA of said expression vehicle is expressed to obtain the protein or polypeptide, and recovering said protein or polypeptide.

11. An antibody to the protein or the polypeptide mentioned in claim 7.

12. The antibody according to claim 11, wherein it is characterized by being a polyclonal antibody.

13. A pharmaceutical composition containing the antibody mentioned in claim 11 as an active ingredient.

14. Vaccine for the prevention of infection of diphtheria containing the protein or the polypeptide mentioned in claim 7 as an active ingredient.

15. A pharmaceutical composition containing the antibody mentioned in claim 12 as an active ingredient.


SEQUENCE LISTING (2 sequences)

SEQ ID NO: 1 - protein, 216 aa, Corynebacterium diphtheriae
MFRRSLASLAAVAVISGVVVAPANAVTVTVNNMTCTIKLTPEETNFIPLSSPVMLTKEKAAFLKNQYGETQLSALDAEIKKKEKELTELEPNAAGREMLTSELKEKKKRFTANKKFSDALDACIAGENYDSSKPNGPKDPNGPKKPGDSDQSGGSEKPNGPKDPGGSEKPNDPKHPEVERALPSTNGAVIGAIVAVLGILAAALPVIKSILRALLP

SEQ ID NO: 2 - DNA, 651 bp, Corynebacterium diphtheriae
atgttcaggcgttcactcgcgtccctcgctgccgtcgctgtcatctctggtgttgttgttgcgccagcgaatgccgtgactgtcacggtcaataatatgacctgcaccattaagctcactcctgaagaaactaattttatacccttgagttcgccggtaatgctcaccaaggaaaaagctgcttttctcaaaaatcaatacggagaaacacagctgtctgcccttgacgcggaaataaagaagaaagaaaaggaactgactgagctcgaacctaacgcggcggggagggagatgttgacgagcgagctgaaagagaaaaagaagaggttcaccgccaacaaaaaattcagtgatgcgctggatgcgtgcatcgcaggtgagaactacgactcgagtaagcccaatggcccaaaggatcccaatggcccgaagaagcccggtgactcggatcagtccggtggctcggagaagcccaatggcccaaaggatcccggtggctcggagaagcccaatgacccgaagcatcccgaagtcgaacgcgctctgccttcgaccaacggagcagtaatcggcgccatcgtcgcggttctcggcatcttggccgccgctctgcctgtcattaagtcgatactgcgggctctcctgccgtaa